(19) United States
(12) Patent Application Publication
(10) Pub. No.: US 2010/0093603 A1
(43) Pub. Date: Apr. 15, 2010

(54) USE OF ORGANIC COMPOUNDS

(76) Inventors: Kenneth Baker, Holland, TX (US); Rajesh Kumar, Temple, TX (US); Vivek Singh, Cincinnati, OH (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/502,418

(22) Filed: Jul. 14, 2009

Related U.S. Application Data
(60) Provisional application No. 61/081,469, filed on Jul. 17, 2008.

(51) Int. Cl. A61K 31/165; A61K 31/4184; A61K 31/55; A61K 38/28; A61P 3/10

(52) U.S. Cl. 514/3; 514/616; 514/381; 514/212.07

(57) ABSTRACT

The invention relates to the use of a renin inhibitor, or a pharmaceutically acceptable salt thereof, alone or in combination with one or more active ingredient, for the manufacture of a medicament for the treatment of diabetic cardiomyopathy.

The enzyme cascade of the renin-angiotensin system (RAS) comprises a series of biochemical events and, as it is well known, there are a variety of approaches for using regulatory intervention to open up treatment possibilities, for example treatment of hypertension. Pharmacological suppression of the RAS, through angiotensin converting enzyme (ACE) inhibition and/or angiotensin receptor blockade, is a proven effective therapeutic approach for the treatment of a wide range of cardiovascular diseases (CVDs). Inhibitors of the enzymatic activity of renin bring about a reduction in the formation of angiotensin I. As a result a smaller amount of angiotensin II is produced. The reduced concentration of that active peptide hormone is the direct cause of, e.g., the antihypertensive effect of renin inhibitors. Accordingly, renin inhibitors, or salts thereof, may be employed, e.g., as antihypertensives or for treating congestive heart failure. Further evaluations may reveal that renin inhibitors may also be employed for a much broader range of therapeutic indications.

As described by Hayat et al (Clinical Science 2004, 1007, 539), diabetic patients have an increased risk of developing heart failure, which is a distinct disease process named diabetic cardiomyopathy. The development of cardiomyopathy represents thus a major complication in patients with diabetes mellitus. There is a need to provide drugs to prevent or delay diabetic cardiomyopathy in diabetic patients. Surprisingly, the present inventors have found that renin inhibitors, such as aliskiren, either alone or in combination, can have a beneficial effect in the treatment of diabetic cardiomyopathy. The present invention provides thus a therapeutic approach for the treatment of diabetic cardiomyopathy.

### SUMMARY OF THE INVENTION

In one aspect, the present invention relates to the use of a renin inhibitor, for example aliskiren, or a pharmaceutically acceptable salt thereof, either alone or in combination with one or more active ingredient, such as, for instance. ACEIs, angiotensin II receptor antagonists, beta blockers, type 2 diabetes therapeutic agents such as a TZDs (thiazolidinediones), type 1 diabetes therapeutic agents such as insulin, or in each case independently a salt thereof, for the manufacture of a medicament for the treatment of diabetic cardiomyopathy.

In one embodiment, the present invention relates to the use of a renin inhibitor, for example aliskiren, or a pharmaceutically acceptable salt thereof, alone for the treatment of diabetic cardiomyopathy. Thus, in one embodiment the renin inhibitor, for example aliskiren, or a pharmaceutically acceptable salt thereof, is administered as monotherapy for the treatment of diabetic cardiomyopathy.

In another aspect, the present invention relates to a pharmaceutical composition for the treatment of diabetic cardiomyopathy, which comprises a renin inhibitor, for example aliskiren, or a pharmaceutically acceptable salt thereof, either alone or in combination with one or more active ingredient, such as, for example, ACEIs, angiotensin II receptor antagonists, beta blockers, type 2 diabetes therapeutic agents such TZDs, type 1 diabetes therapeutic agents such as insulin, or in each case independently a salt thereof.

In still another embodiment, the present invention relates to a pharmaceutical composition for the treatment of diabetic cardiomyopathy, which comprises a renin inhibitor, for example aliskiren, or a pharmaceutically acceptable salt thereof, without any further active ingredient. Thus, in one embodiment the renin inhibitor, for example aliskiren, or a pharmaceutically acceptable salt thereof, is administered as monotherapy for the treatment of diabetic cardiomyopathy.

In a further embodiment, the present invention relates to a pharmaceutical composition for simultaneous, separate or sequential use for the treatment of diabetic cardiomyopathy, comprising a renin inhibitor, for example aliskiren, or a pharmaceutically acceptable salt thereof, in combination with one or more active ingredient e.g. selected from the group consisting of ACE inhibitors, angiotensin II receptor antagonists, beta-blockers, type 2 diabetes therapeutic agents such as TZDs, and type 1 diabetes therapeutic agents such as insulin, or in each case independently a salt thereof, in each case in a unit dosage form, in admixture with a pharmaceutically acceptable carrier.

The invention furthermore relates to a method for the treatment of diabetic cardiomyopathy, which comprises administering to a warm-blooded animal, including human, a therapeutically effective amount of a renin inhibitor, for example aliskiren, or a pharmaceutically acceptable salt thereof, either alone or in combination with one or more active ingredient, such as, for example, ACEIs, beta blockers, angiotensin II receptor antagonist, type 2 diabetes therapeutic agents such as TZDs, type 1 diabetes therapeutic agents such as insulin, or in each case independently a salt thereof.

### EMBODIMENTS ACCORDING TO THE INVENTION

The renin inhibitors to which the present invention applies are any of those having renin inhibitory activity in vivo and, therefore, pharmaceutical utility, such as therapeutic agents for the prevention of, delay the onset of and/or treatment of e.g., hypertension (whether, for example, for malignant, essential, isolated systolic, or other secondary type of hypertension). Further indications for which renin inhibitors can be useful are described e.g. in WO2004/002549, WO2005/089731, WO2006/041763, WO2006/041974, WO2006/116435, WO2002/40007 and in PCT application No. 2007/065564.

For example, the present invention relates to renin inhibitors selected from the group consisting of the renin inhibitors disclosed in:

U.S. Pat. Nos. 5,559,111, 6,197,959, 6,376,672, 6,051,712, 6,197,959, 6,268,499 and 6274735; in US patent applications 2004/0204455, 2002/087002; in WO2003/099767, WO2005/054177, WO2005/051895, WO2005/051911, WO2006/066896, WO2006/069788, WO2006/074924, WO2006/094763, WO2006/100036, WO2006/0117183, WO2006/125621, WO2006/128659, WO2007/006534, WO2007/077005; in WO2003/093267, WO2004/002957, WO2004/096116, WO2004/096799, WO2004/096803, WO2004/096804, WO2005/040120, WO2005/040165, WO2005/040173, WO2005/054243, WO2005/054244, WO2006/021399, WO2006/021401, WO2006/021402, WO2006/021403, WO2006/058546, WO2006/059304, WO2006/061791, WO2006/063610, WO2006/064484, WO2006/079988, WO2006/092268, WO2006/129237, WO2006/131884, WO2007/034406, WO2007/034445, WO2007/049224, WO2003/103652, WO2003/103652, WO2007/045551, WO2004/089915, WO2000/63173, WO2000/64873, WO2000/64887, WO1997/09311, WO2005/037803, WO2005/061457, WO2005/070870, WO2005/070871, WO2005/070877, WO2005/090304, WO2005/090305, WO2006/005741, WO2006/061426, WO2006/061427, WO2006/095020, WO2006/103273, WO2006/103275, WO2006/103277, WO2007/031557 and WO2007/031558; in PCT applications 2007/005130 and 2007/005131; in European patent application 07111290.8 and in European patent No. 0863875;

in particular in the compound claims and the final products of the working examples.

Renin inhibitors are selected, for example, from the group consisting of ditekiren, terlakiren, zankiren, aliskiren and salts thereof. In one embodiment, the renin inhibitor is aliskiren or a salt thereof, such as the hemi-fumarate, nitrate, hydrogen sulfate and orotate, in particular the hemi-fumarate salt thereof. Aliskiren in form of the free base is chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide and is specifically disclosed in EP 678503 A as Example 83.

In another embodiment, the present invention relates to the simultaneous, separate or sequential use of a renin inhibitor or a pharmaceutically acceptable salt thereof in combination with one or more active ingredient for the manufacture of a medicament for the treatment of diabetic cardiomyopathy.

In one embodiment, other active ingredients are selected from the group consisting of ACEIs, beta blockers, angiotensin II receptor antagonists, type 2 diabetes therapeutic agents such as TZD, and type 1 diabetes therapeutic agents such as insulin, or in each case independently a salt thereof.

In another embodiment, other active ingredients to be used in a combination with renin inhibitors, or salts thereof, are ACE inhibitors, or salts thereof.

Suitable ACEIs which may be employed according to the present invention include ACEIs having differing structural features, for example a member of the group consisting of alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, trandolapril and salts thereof. In one embodiment, the ACE inhibitor is selected from the group consisting of benazepril, benazeprilat, captopril, enalapril, enaprilat and salts thereof; in another embodiment the ACE inhibitor is benazepril, benazeprilat or salts thereof.

In yet another embodiment, the present invention relates to the simultaneous, separate or sequential use of aliskiren, or a salt thereof, in combination with an ACEI, or salt thereof, such as alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, trandolapril or salts thereof, in particular benazepril, benazeprilat, captopril, enalapril, enaprilat or salts thereof, particularly benazepril, benazeprilat or salts thereof.

In a still further embodiment, the present invention relates to the simultaneous, separate or sequential use of a renin inhibitor or a pharmaceutically acceptable salt thereof in combination with an angiotensin II receptor antagonist for the manufacture of a medicament for the treatment of diabetic cardiomyopathy.

Suitable angiotensin II receptor antagonists which may be employed according to the present invention include angiotensin II receptor antagonists having differing structural features. For example, mention may be made of the compounds which are listed in the European Patent Application having the No. 443983 and in the European Patent No. 1146872B1; in particular in the compound claims and the final products of the working examples, for example, (S)—N-(1-carboxy-2-methylprop-1-yl)-N-pentanoyl-N-[2′(1H-tetrazol-5-yl)biphenyl-4-ylmethyl]amine [Valsartan] and its pharmaceutically acceptable salts. Furthermore, reference is made to the compounds which are listed in: European Patent Application having the publication No. 253310, European Patent Application having the publication No. 403159, PCT Patent Application having the publication No. WO 91/14679, European Patent Application having the publication No. 420237, European Patent Application having the publication No. 502314, European Patent Application having the publication No. 459136, European Patent Application having the publication No. 504888, European Patent Application having the publication No. 514198, European Patent Application having the publication No. 475206, PCT Patent Application having the publication No. WO 1993/20816; in particular in the compound claims and the final products of the working examples therein. In one embodiment, the angiotensin II receptor antagonists selected from the group consisting of valsartan, losartan, eprosartan, irbesartan, telmisartan, candesartan, saprisartan, olmesartan and salts thereof; preferably valsartan, losartan, eprosartan, irbesartan, telmisartan, candesartan, saprisartan and salts thereof; in particular the angiotensin II receptor antagonists is valsartan or salt thereof.

In a particular embodiment, the present invention relates to the simultaneous, separate or sequential use of aliskiren, or a salt thereof, in combination with an angiotensin II receptor antagonist, or a salt thereof, such as valsartan, losartan, eprosartan, irbesartan, telmisartan, candesartan, saprisartan or salts thereof; particularly valsartan or salt thereof.

In a yet further embodiment, the present invention relates to the simultaneous, separate or sequential use of a renin inhibitor or a pharmaceutically acceptable salt thereof in combination with a β-blocker for the manufacture of a medicament for the treatment of diabetic cardiomyopathy.

β-blockers suitable for use in the present invention include β-adrenergic blocking agents (β-blockers) which compete with epinephrine for β-adrenergic receptors and interfere with the action of epinephrine. In particular, the β-blockers are selective for the β-adrenergic receptor as compared to the alpha (α)-adrenergic receptors, and so do not have a significant α-blocking effect. Suitable β-blockers include, for example, compounds selected from acebutolol, atenolol, betaxolol, bisoprolol, carteolol, carvedilol, esmolol, labetalol, metoprolol, nadolol, oxprenolol, penbutolol, pindolol, propranolol, sotalol, timolol and salts thereof; particularly atenolol, metoprolol, propranolol and salts thereof. In one embodiment, β-blockers for use in the present invention are atenolol, metoprolol, propranolol and salts thereof.

In still another embodiment, the present invention relates to the simultaneous, separate or sequential use of a renin inhibitor (eg. aliskiren) or a pharmaceutically acceptable salt thereof in combination with a type 2 diabetes therapeutic agent, such as TZDs, for the manufacture of a medicament for the treatment of diabetic cardiomyopathy.

TZDs suitable for use in the present invention include, for example, compounds selected from thiazolidinediones (TZDs), including troglitazone, rosiglitazone, ciglitazone; darglitazone; englitazone; isaglitazone, pioglitazone, and salts thereof. In one embodiment, the TZD is selected from troglitazone, rosiglitazone, pioglitazone, and salts thereof.

In still another embodiment, the present invention relates to the simultaneous, separate or sequential use of a renin inhibitor (eg. aliskiren) or a pharmaceutically acceptable salt thereof in combination with a type 1 diabetes agent, such as insulin, or salt thereof, for the manufacture of a medicament for the treatment of diabetic cardiomyopathy.

In a further embodiment, the present invention relates to a pharmaceutical composition for the treatment of diabetic cardiomyopathy comprising a renin inhibitor, such as aliskiren, or a pharmaceutically acceptable salt thereof, in combination with an ACE inhibitor or salt thereof. ACEIs of this embodiment are selected, for example, from the group of ACEIs above mentioned; the ACEI is, for example, benazepril or salt thereof.

In a still further embodiment, the present invention relates to a pharmaceutical composition for the treatment of diabetic cardiomyopathy comprising a renin inhibitor, such as aliskiren, or a pharmaceutically acceptable salt thereof, in combination with an angiotensin II receptor antagonist or salt thereof. Angiotensin II receptor antagonists of this embodiment are selected, for example, from the group of angiotensin II receptor antagonists above mentioned; the angiotensin II receptor antagonist is for example, valsartan or salt thereof.

In a yet further embodiment, the present invention relates to a pharmaceutical composition for the treatment of diabetic cardiomyopathy comprising a renin inhibitor, such as aliskiren, or a pharmaceutically acceptable salt thereof, in combination with a β-blocker or salt thereof. β-blockers of this embodiment are selected, for example, from the group of β-blockers above mentioned.

In a even further embodiment, the present invention relates to a pharmaceutical composition for the treatment of diabetic cardiomyopathy comprising a renin inhibitor, such as aliskiren, or a pharmaceutically acceptable salt thereof, in combination with a type 2 diabetes therapeutic agent, such as TZD, or salt thereof. TZDs of this embodiment are selected, for example, from the group of TZDs above mentioned.

In yet a further embodiment, the present invention relates to a pharmaceutical composition for the treatment of diabetic cardiomyopathy comprising a renin inhibitor, such as aliskiren, or a pharmaceutically acceptable salt thereof, in combination with a type 1 diabetes therapeutic agent, such as insulin, or salt thereof.

In yet another embodiment, the present invention relates to a method for treatment of diabetic cardiomyopathy, which method comprises administering to a warm-blooded animal, including man, in need thereof, a therapeutically effective amount of a pharmaceutical composition comprising a renin inhibitor, such as aliskiren, or a pharmaceutically acceptable salt thereof, in combination with an ACEI, or salt thereof. ACEIs of this embodiment are selected, for example, from the group of ACEIs above mentioned; the ACEI is, for example, benazepril or salt thereof.

In another embodiment, the present invention relates to a method for treatment of diabetic cardiomyopathy, which method comprises administering to a warm-blooded animal, including man, in need thereof, a therapeutically effective amount of a pharmaceutical composition comprising a renin inhibitor, such as aliskiren, or a pharmaceutically acceptable salt thereof, in combination with an angiotensin II receptor antagonist, or a salt thereof. Angiotensin II receptor antagonists of this embodiment are selected, for example, from the group of angiotensin II receptor antagonists above mentioned; the angiotensin II receptor antagonist is, for example, valsartan or salt thereof.

In still another embodiment, the present invention relates to a method for treatment of diabetic cardiomyopathy, which method comprises administering to a warm-blooded animal, including man, in need thereof, a therapeutically effective amount of a pharmaceutical composition comprising a renin inhibitor, such as aliskiren, or a pharmaceutically acceptable salt thereof, in combination with a β-blocker, or salt thereof. β-blockers of this embodiment are selected, for example, from the group of β-blockers above mentioned.

In yet another embodiment, the present invention relates to a method for treatment of diabetic cardiomyopathy, which method comprises administering to a warm-blooded animal, including man, in need thereof, a therapeutically effective amount of a pharmaceutical composition comprising a renin inhibitor, such as aliskiren, or a pharmaceutically acceptable salt thereof, in combination with a type 2 diabetes therapeutic agent, such as TZD, or salt thereof. TZDs of this embodiment are selected, for example, from the group of TZDs above mentioned.

In yet another embodiment, the present invention relates to a method for treatment of diabetic cardiomyopathy, which method comprises administering to a warm-blooded animal, including man, in need thereof, a therapeutically effective amount of a pharmaceutical composition comprising a renin inhibitor, such as aliskiren, or a pharmaceutically acceptable salt thereof, in combination with a type 1 diabetes therapeutic agent, such as insulin, or salt thereof.

Listed below are definitions of various terms used throughout the specification:

The term “aliskiren”, if not defined specifically, is to be understood both as the free base and as a salt thereof, especially the hemi-fumarate, nitrate, hydrogen sulfate and orotate salt thereof, in particular the hemi-fumarate salt thereof.

The term “valsartan”, if not defined specifically, is to be understood both as the free base and as a salt thereof, especially a pharmaceutically acceptable salt thereof, as described below.

Valsartan , or a pharmaceutically acceptable salt thereof, can, e.g., be prepared in a manner known per se. Salts forms include acid addition salts. The compounds having at least one acid group (e.g., COOH or 5-tetrazolyl) can also form salts with bases. Suitable salts with bases are, e.g., metal salts, such as alkali metal or alkaline earth metal salts, e.g., sodium, potassium, calcium or magnesium salts, or salts with ammonia or an organic amine, such as morpholine, thiomorpholine, piperidine, pyrrolidine, a mono-, di- or tri-lower alkylamine, e.g., ethyl-, tert-butyl-, diethyl-, diisopropyl-, triethyl-, tributyl- or dimethylpropylamine, or a mono-, di- or trihydroxy lower alkylamine, e.g., mono-, di- or tri-ethanolamine. Corresponding internal salts may furthermore be formed. Salts which are unsuitable for pharmaceutical uses but which can be employed, e.g., for the isolation or purification of free compounds I or their pharmaceutically acceptable salts, are also included. In one embodiment, salts are, e.g., selected from the mono-sodium salt in amorphous form; di-sodium salt of Valsartan in amorphous or crystalline form, especially in hydrate form, thereof.

Mono-potassium salt of Valsartan in amorphous form; di-potassium salt of Valsartan in amorphous or crystalline form, especially in hydrate form, thereof.

Calcium salt of Valsartan in crystalline form, especially in hydrate form, primarily the tetrahydrate thereof; magnesium salt of Valsartan in crystalline form, especially in hydrate form, primarily the hexahydrate thereof; calcium/magnesium mixed salt of Valsartan in crystalline form, especially in hydrate form; bis-diethylammonium salt of Valsartan in crystalline form, especially in hydrate form; bis-dipropylammonium salt of Valsartan in crystalline form, especially in hydrate form; bis-dibutylammonium salt of Valsartan in crystalline form, especially in hydrate form, primarily the hemihydrate thereof; mono-L-arginine salt of Valsartan in amorphous form; bis-L-arginine salt of Valsartan in amorphous form; mono-L-lysine salt of Valsartan in amorphous form; bis-L-lysine salt of Valsartan in amorphous form.

In one embodiment, Valsartan is used as the free acid.

Where the β-blocker is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug, such as a physiologically hydrolizable and acceptable ester. For example, metoprolol is suitably administered as its tartrate salt, propranolol is suitably administered as the hydrochloride salt, and so forth.

Type 2 and type 1 diabetes therapeutic agents refer to anti-diabetic agents and include, for example, insulin, insulin derivatives and mimetics; insulin secretagogues such as the sulfonylureas e.g. glisoxepid, glyburide, glibenclamide, acetohexamide, chloropropamide, glibornuride, tolbutamide, tolazamide, glipizide, carbutamide, gliquidone, glyhexamide, phenbutamide or tolcyclamide; and preferably glimepiride, gliclazide, and Amaryl; insulinotropic sulfonylurea receptor ligands such as meglitinides, e.g., nateglinide and repaglinide; antidiabetic D-phenylalanine derivative; thiazolidinedione derivatives such as glitazones, e.g., pioglitazone, troglitazone or rosiglitazone, in particular pioglitazone or rosiglitazone; glucokinase activators; protein tyrosine phosphatase-1B (PTP-1B) inhibitors such as PTP-112; agonists of Beta-3 AR (adrenergic receptor), agonists of UCP (uncoupling protein), inhibitors of PEPCK (phosphoenolpyruvate carboxykinase), PDHK (pyruvate dehydrogenase kinase) inhibitors, GSK3 (glycogen synthase kinase-3) inhibitors such as SB-517955, SB-4195052, SB-216763, NN-57-05441 and NN-57-05445; inhibitors of GFAT (glutamine fructose-6-phosphate amidotransferase), inhibitors of G6Pase (glucose-6-phosphatase), inhibitors of F-1,6-BPase (fructose-1,6-bisphosphatase), inhibitors of GP (glycoprotein), RXR (retinoid X receptor) ligands or agonists such as GW-0791 and AGN-194204; sodium-dependent glucose co-transporter inhibitors such as T-1095; glycogen phosphorylase A inhibitors such as BAY R3401; aldose reductase inhibitors; sorbitol dehydrogenase inhibitors; biguanides such as metformin; alpha-glucosidase inhibitors such as acarbose, adiposine, voglibose, miglitol, emiglitate, camiglibose, tendamistate, trestatin, pradimicin-Q and salbostatin; glucagon receptor antagonists, inhibitors of GSK-3, GLP-1 (glucagon like peptide-1). GLP-1 analogs such as Exendin-4 and GLP-1 mimetics; GLP-1 agonists; modulators of PPARs (peroxisome proliferator-activated receptors), e.g., non-glitazone type PPARγ agonists such as N-(2-benzoylphenyl)-L-tyrosine analogues, e.g. GI-262570, and JTT501 or dual PPARγ/PPARα agonists; antidiabetic vanadium; antidiabetic phenylacetic acid derivative, β-cell imidazoline receptor antagonists, Estrogen-related receptor gamma (ERRγ) agonist e.g. GSK4716 or GSK9089; agonists or antagonists of the estrogen-related receptors (ERR) e.g. of the ERRα, ERRβ, and ERRγ receptors; DPPIV (dipeptidyl peptidase IV) inhibitors such as (S)-1-[(3-hydroxy-1-adamantyl)amino]acetyl-2-cyano-pyrrolidine (also known as LAF237 or vildagliptin) of formula

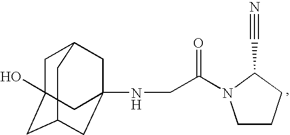

and L-threo-isoleucyl thiazolidine (compound code according to Probiodrug: P32/98 as described above), Sitagliptin (also known as MK-0431), (2S)-1-{(2-(5-Methyl-2-phenyl-oxazol-4-yl)-ethylamino)-acetyl}-pyrrolidine-2-carbonitrile, or (2S)-1-{(1,1-Dimethyl-3-(4-pyridin-3-yl-imidazol-1-yl)-propylamino)-acetyl}-pyrrolidine-2-carbonitrile, (S)-1-((2S,3S,11bS)-2-Amino-9,10-dimethoxy-1,3,4,6,7,11b-hexahydro-2H-pyrido (2,1-a) isoquinolin-3-yl)-4-fluoromethyl-pyrrolidin-2-one, or (S,S,S,S)-1-(2-Amino-9,10-dimethoxy-1,3,4,6,7,1 11b-hexahydro-2H-pyrido (2,1-a) isoquinolin-3-yl)-4-methyl-pyrrolidin-2-one, GSK23A, saxagliptin, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide; SCD-1 (stearoyl-CoA desaturase-1) inhibitors; DGAT1 and DGAT2 (diacylglycerol acyltransferase 1 and 2) inhibitors; ACC2 (acetyl CoA carboxylase 2) inhibitors; inhibitors of protein tyrosine phosphatases (PTPases), in particular, inhibitors of PTPase-1B (PTP-1B) and T-cell PTPase (TC PTP), inhibitors of gastric emptying, and breakers of AGE (advanced glycation end products), or in any case a pharmaceutically accepted salt thereof.

Where the type 2 diabetes therapeutic agent, such as a TZD (thiazolidinedione), is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug, such as a physiologically hydrolizable and acceptable ester.

Salts are especially the pharmaceutically acceptable salts. They can be formed where salt forming groups, such as basic or acidic groups, are present that can exist in dissociated form at least partially, e.g. in a pH range from 4 to 10 in aqueous solutions, or can be isolated especially in solid, especially crystalline, form. Such salts are formed, for example, as acid addition salts, for example with organic or inorganic acids, from compounds with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzene-sulfonic acid, 2-naphthalenesulfonic acid, 1,5-naphthalene-disulfonic acid, N-cyclohexyl-sulfamic acid, N-methyl-, N-ethyl- or N-propyl-sulfamic acid, or other organic protonic acids, such as ascorbic acid. In the presence of negatively charged radicals, such as carboxy or sulfonyl, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-di-methylpiperazine. When a basic group and an acid group are present in the same molecule, a compound may also form internal salts.

The active agents may be present in prodrug form. The invention includes prodrugs for the active pharmaceutical species of the invention, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), each of which is incorporated herein by reference.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned:

Oxidative Activation
N- and O-dealkylation
Oxidative deamination
N-oxidation
Epoxidation

Reductive Activation
Azo reduction
Sulfoxide reduction
Disulfide reduction
Bioreductive alkylation
Nitro reduction.

Also to be mentioned as metabolic activations of prodrugs are nucleotide activation, phosphorylation activation and decarboxylation activation. For additional information, see “The Organic Chemistry of Drug Design and Drug Action”, R B Silverman (particularly Chapter 8, pages 497 to 546).

Although protected derivatives of compounds of the invention may not possess pharmacological activity as such, they may be administered, for example parenterally or orally, and thereafter metabolised in the body to form compounds of the invention which are pharmacologically active. Such derivatives are therefore examples of “prodrugs”. All prodrugs of the described compounds are included within the scope of the invention. The use of protecting groups is fully described in ‘Protective Groups in Organic Chemistry’, edited by J W F McOmie, Plenum Press (1973), and ‘Protective Groups in Organic Synthesis’, 2nd edition, T W Greene & P G M Wutz, Wiley-Interscience (1991).

Where the plural form is used for compounds, salts, pharmaceutical compositions, diseases, disorders and the like, this is intended to mean one or more single compound(s), salt(s), pharmaceutical composition(s), disease(s), disorder(s) or the like, where the singular or the indefinite article (“a”, “an”) is used, this is intended to include the plural (for example also different configuration isomers of the same compound, e.g. enantiomers in racemates or the like) or the singular (“one”).

The terms “effective amount” or “therapeutically effective amount” refers to the amount of the active ingredient or agent which halts or reduces the progress of diabetic cardiomyopathy, or which otherwise completely or partly cures or acts palliatively on the condition.

The term “prophylactically effective amount” refers to the amount of the active ingredient or agent prevents the onset of diabetic cardiomyopathy.

The terms “drug”, “active substance”, “active ingredient”, “active agent” are to be understood as meaning a compound in free form or in the form of a pharmaceutically acceptable salt, in particular compounds of the type specified herein.

The term “warm-blooded animal or patient” are used interchangeably herein and include, but are not limited to, humans, dogs, cats, horses, pigs, cows, monkeys, rabbits, mice and laboratory animals. In one embodiment, the mammals are humans.

The term “treatment” means the management and care of a patient for the purpose of preventing, combating or delaying progression of the disease, condition or disorder, preferably for the purpose of combating the disease, condition or disorder, and in particular it also prophylactic treatment.

The terms “prevention”/“preventing” are to be understood as meaning the prophylactic administration of a drug, such as a combined preparation or pharmaceutical composition, to healthy patients to prevent the outbreak of the disease, condition or disorder.

The terms “delay of progression”/“delaying progression” are to be understood as meaning the administration of a drug, such as a combined preparation or pharmaceutical composition, to patients being in a pre-stage of the disease, condition or disorder.

The term “diabetes” embraces both type 1 and type 2 diabetes. The term “type 1 diabetes” refers to insulin dependent diabetes mellitus (IDDM), which is a chronic autoimmune disease in which insulin-producing cells (P cells) within the pancreatic islets of Langerhan are selectively targeted and destroyed by an infiltrate of immunological cells. IDDM is characterized by a progressive loss of pancreatic beta cells due to an unfavorable balance between the destructive autoimmune processes targeting beta cells on the one hand and the regenerative capacity of these cells on the other hand. This imbalance eventually leads to total loss of beta cells and endogenous insulin secretion. The term “type 2 diabetes” means type 2 diabetes mellitus and it is a disease in which the pancreas does not secrete sufficient insulin due to an impairment of pancreatic beta (β)-cell function and/or in which there is insensitivity to produce insulin (insulin resistance). Typically, the fasting plasma glucose is less than 126 mg/dL, while pre-diabetes is, e.g., a condition which is characterized by one of following conditions: impaired fasting glucose (110-125 mg/dL) and impaired glucose tolerance (fasting glucose levels less than 126 mg/dL and post-prandial glucose level between 140 mg/dL and 199 mg/dL). Type 2 diabetes mellitus can be associated with or without hypertension. Diabetes mellitus occurs frequently, e.g., in African American, Latino/Hispanic American, Native American, Native American, Asian American and Pacific Islanders. Markers of insulin resistance include HbA1C, HOMA IR, measuring collagen fragments. TGF-β in urine. PAI-1 and prorenin.

Diabetic patients have an increased risk of developing heart failure, which is a distinct disease process named “diabetic cardiomyopathy”. The term “diabetic cardiomyopathy” (as defined by Hayat et al in Clinical Science 2004, 1007, 539) is a disease process which affects the myocardium in diabetic patients causing a wide range of structural abnormalities eventually leading to LVH [left ventricular hypertrophy] and diastolic and systolic dysfunction or a combination of these. Diabetic cardiomyopathy is characterized by myocellular hypertrophy and myocardial fibrosis (Bell, Diabetes Care, 2003, 26, 2433). As described by Scognamiglio (The American Journal of Cardiology, 2004, 93, 17A), diabetic cardiomyopathy is a condition characterized by defects of the contractile function in the absence of significant coronary artery disease or systemic hypertension. As pointed out by Bell (Diabetes Care, 2003, 26, 2433), the epidemiology of heart failure (HF) in diabetic patients can be summarized as follows:

1) HF is two times as common in diabetic men and five times as common in diabetic women as in age-matched non-diabetic subjects.

2) About 3.3% of type 2 diabetic subjects develop HF each year.

3) Elderly diabetic subjects have a 1.3-fold greater risk of developing HF than nondiabetic subjects.

4) Prevalence of HF in elderly diabetic subjects is 39%.

5) 1% rise in HbA1c is associated with a 15% increased risk of HF in elderly diabetic patients.

6) Diabetic patients account for 25% of all patients enrolled in large HF trials.

The structure of the active agents identified by generic or tradenames or code numbers may be taken from the actual edition of the standard compendium “The Merck Index” or from databases, e.g., Life Cycle Patents International (e.g. IMS World Publications). Any person skilled in the art is fully enabled to identify the active agents and, based on these references, likewise enabled to manufacture and test the pharmaceutical indications and properties in standard test models, both in vitro and in vivo.

The pharmaceutical compositions according to the invention are those suitable for enteral, such as oral or rectal, transdermal and parenteral administration to mammals, including man, with the compositions comprising the pharmacological active compound either alone or together with customary pharmaceutical auxiliary substances. For example, the pharmaceutical compositions consist of from about 0.1% to 100%, such as of from about 1% to about 80%, of the active compound. Pharmaceutical compositions for enteral or parenteral administration are, for example, in unit dose forms, such as coated tablets, tablets, capsules or suppositories and also ampoules. These are prepared in a manner which is known per se, for example using conventional mixing, granulation, coating, solubilizing or lyophilizing processes. Thus, pharmaceutical compositions for oral use can be obtained by combining the active compound with solid excipients, if desired granulating a mixture which has been obtained, and, if required or necessary, processing the mixture or granulate into tablets or coated tablet cores after having added suitable auxiliary substances. For oral administration, the pharmaceutical composition comprising a renin inhibitor, in particular, aliskiren, for example in the form of the hemi-fumarate salt thereof; and optionally at least one therapeutic agent selected from the group consisting of an ACE inhibitor, an angiotensin II receptor antagonist, a beta-blocker, a type 2 diabetes therapeutic agent, such as a TZD, a type 1 diabetes therapeutic agent, such as insulin, and pharmaceutically acceptable salts thereof, can take the form, for example, of solutions, suspensions, tablets, pills, capsules, powders, microemulsions and unit dose packets. In one embodiment, the pharmaceutical composition is in the form of tablets or gelatin capsules comprising the active ingredient together with: a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbants, colorants, flavors and sweeteners. Injectable compositions are, for example, aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers.

The dosage of the active compound can depend on a variety of factors, such as mode of administration, homeothermic species, age and/or individual condition. In particular, dosages for pharmaceutical combinations are therapeutically effective dosages, especially those which are commerically available. In one embodiment, the doses are low dose combinations.

Normally, in the case of oral administration, an approximate daily dose of from 1 mg to about 360 mg is to be estimated, e.g., for a patient of approximately 75 kg in weight.

Usually, children receive about half of the adult dose or they can receive the same dose as adults. The dose necessary for each individual can be monitored, e.g., by measuring the serum concentration of the active ingredient, and adjusted to an optimum level. All doses are based on the active agent, i.e. for aliskiren, the doses are based on the free base.

The doses of renin inhibitor, for example aliskiren, to be administered to warm-blooded animals, for example human beings, of, for example, approximately 70 kg body weight, especially the doses effective for the inhibition of the enzyme renin, e.g. in lowering blood pressure, may be of from 3 mg to 3 g, such as of from 10 mg to 1 g, for example of from 20 mg to 600 mg (e.g. 150 mg to 300 mg), per person per day; divided, for example, into 1 to 4 single doses which may, e.g., be of the same size. Single doses, of for example aliskiren, comprise, for example, 75, 100, 150, 200, 250, 300 or 600 mg per adult patient.

In one embodiment, dosage unit forms of angiotensin II receptor antagonist may be, for example, tablets or capsules comprising e.g. a therapeutically effective amount, e.g. of from 10 to about 360 mg of, for example, valsartan, in particular 40 mg, 80 mg, 160 mg or 320 mg. The application of the active ingredient may occur up to three times a day, starting e.g. with a daily dose of 20 mg or 40 mg of, for example, valsartan, increasing via 80 mg daily and further to 160 mg daily up to 320 mg daily. In one embodiment, valsartan is applied twice a day with a dose of 80 mg or 160 mg, respectively, each. Corresponding doses may be taken, for example, in the morning, at mid-day or in the evening. In one embodiment, administration is b.i.d.

Dosage unit forms of ACE inhibitors are, for example, tablets or capsules comprising e.g. of from 5 mg to 20 mg, such as 5 mg, 10 mg or 20 mg of, for example, benazepril; of from 6.5 mg to 100 mg, such as 6.25 mg, 12.5 mg, 25 mg, 50 mg, 75 mg or 100 mg of, for example, captopril; of from 2.5 mg to 20 mg, such as 2.5 mg, 5 mg, 10 mg or 20 mg of, for example, enalapril; of from 10 mg to 20 mg, such as 10 mg or 20 mg of, for example, fosinopril; of from 2.5 mg to 4 mg, such as 2 mg or 4 mg of, for example, perindopril; of from 5 mg to 20 mg, such as 5 mg, 10 mg or 20 mg of, for example, quinapril; or of from 1.25 mg to 5 mg, such as 1.25 mg, 2.5 mg, or 5 mg of, for example, ramipril. In one embodiment, administration is t.i.d.

Suitable daily dosages of β-blockers (for adults) for oral administration are, for example: of from 200 to 1200 mg of, for example, acebutolol; of from 25 to 100 mg of, for example, atenolol; of from 10 to 20 mg of, for example, betaxolol; of from 5 to 10 mg of, for example, bisoprolol; of from 2.5 to 10 mg of, for example, carteolol; of from 100 to 1,800 mg of, for example, labetalol; of from 50 to 450 mg of, for example, metoprolol; of from 40 to 240 mg of, for example, nadolol; of from 60 to 480 mg of, for example, oxprenolol; of from 20 to 80 mg of, for example, penbutolol; of from 10 to 60 mg of, for example pindolol; of from 40 to 320 mg or of from 60 to 320 mg (for long-acting formulation) of, for example, propranolol; of from 160 to 320 mg of, for example, sotalol; of from 20 to 60 mg of, for example, timolol.

Suitable daily dosages of a type 2 diabetes therapeutic agent, such as a TZD, for oral administration are, for example: of from 0.001 mg/kg to about 100 mg/kg, such as of from 0.01 mg to 2000 mg per day. e.g. 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850, 1,000 and 2,000 milligrams.

Ultimately, the exact dose of the active agent and the particular formulation to be administered depend on a number of factors, e.g., the rate of release of the active agent. For example, the amount of the active agent required and the release rate thereof may be determined on the basis of known in vitro or in vivo techniques, determining how long a particular active agent concentration in the blood plasma remains at an acceptable level for a therapeutic effect.

In particular, for those active ingredients of the pharmaceutical combination according to the present invention that are commercially available, are especially therapeutically effective commercially available dosages.

For the larger mammals, an indicated total daily dosage is in the range from about 0.01 to 100 mg/kg of the compound, conveniently administered in divided doses 1 to 4 times a day in unit dosage form containing for example from about 0.1 to about 400 mg of the compound in sustained release form.

Since the present invention has an aspect that relates to methods for treatment with a combination of compounds which may be administered separately, the invention also relates to combining separate pharmaceutical compositions in a kit form. The kit may comprise, e.g., two or three separate pharmaceutical compositions: (1) a composition comprising a renin inhibitor, or a pharmaceutically acceptable salt thereof, and a pharmaceutically acceptable carrier or diluent and (2) at least one therapeutic agent selected from the group consisting of an ACE inhibitor, or a pharmaceutically acceptable salt thereof, an angiotensin II receptor antagonist, e.g., valsartan or a pharmaceutically acceptable salt thereof, a beta blocker or a pharmaceutically acceptable salt thereof, a type 2 diabetes therapeutic agent, such as a TZD, or a pharmaceutically acceptable salt thereof, a type 1 diabetes therapeutic agent, such as insulin, or a pharmaceutically acceptable salt thereof, and a pharmaceutically acceptable carrier or diluent. The amounts of (1) and (2) are such that, when co-administered separately a beneficial therapeutic effect(s) is achieved. The kit comprises a container for containing the separate compositions such as a divided bottle or a divided foil packet, wherein each compartment contains a plurality of dosage forms (e.g., tablets) comprising, e.g., (1) or (2). Alternatively, rather than separating the active ingredient-containing dosage forms, the kit may contain separate compartments each of which contains a whole dosage which in turn comprises separate dosage forms. An example of this type of kit is a blister pack wherein each individual blister contains two or three (or more) tablets, one (or more) tablet(s) comprising a pharmaceutical composition (1), and the second (or more) tablet(s) comprising a pharmaceutical composition (2). Typically the kit comprises directions for the administration of the separate components. The kit form is particularly advantageous when the separate components are administered in different dosage forms (e.g., oral and parenteral), are administered at different dosage intervals, or when titration of the individual components of the combination is desired by the prescribing physician. In the case of the instant invention a kit may, e.g., comprise:

(1) a therapeutically effective amount of a composition comprising a renin inhibitor, in particular, aliskiren, for example in the form of the hemi-fumarate salt thereof, and a pharmaceutically acceptable carrier or diluent, in a first dosage form, and
(2) at least one therapeutic agent selected from the group consisting of an ACE inhibitor or a pharmaceutically acceptable salt thereof, an angiotensin II receptor antagonist (e.g., valsartan) or a pharmaceutically acceptable salt thereof, a beta blocker or a pharmaceutically acceptable salt thereof, a type 2 diabetes therapeutic agent, such as a TZD, or a pharmaceutically acceptable salt thereof, a type 1 diabetes therapeutic agent, such as insulin, or a pharmaceutically acceptable salt thereof, and a pharmaceutically acceptable carrier or diluent, in a second dosage form, and
(3) a container for containing said first and second and dosage forms.

As described by Bell (Diabetes Care, 2003, 26, 2433), diabetic cardiomyopathy is characterized by myocellular hypertrophy and myocardial fibrosis. The etiology of diabetic cardiac dysfunction is thus multiparametric. The study as described herein to show the suitability of a renin inhibitor such as aliskiren in the treatment of diabetic cardiomyopathy focuses on the analysis of the markers of cardiac dysfunction, in streptozotocin-induced diabetic mice. The following parameters are analyzed:

1) Analysis of morphometric and hemodynamic parameters: analysis performed by echocardiography and LV catheterization identifies the effect of the test agents on cardiac pathology, such as diastolic and systolic dysfunction, caused by diabetes.

2) Analysis of enhanced reactive oxygen species (ROS) generation, myocyte apoptosis, and cardiac fibrosis by histology and gene expression: ROS generation, apoptosis, and fibrosis are detected by immunohistochemistry using specific staining and by measuring expression of genes (such as NADPH oxidase, Bcl2, Bax, TGF-β, and collagen), which contribute to these conditions. These measurements identify the effect of the test agents on cardiac fibrosis, apoptosis, and ROS production. Production of ROS has been proposed as the main causative factor of diabetic cardiac dysfunction, which also results in cardiac myocyte apoptosis. Cardiac fibrosis is considered to contribute to diastolic dysfunction.

3) Analysis of the cardiac RAS by Ang II measurement and gene expression: this measurement identifies the effect of the test agents on activation of the cardiac RAS in diabetes. The RAS is implicated in the pathogenesis of diabetic cardiac dysfunction. Activation of the intracellular RAS in vitro and in vivo has been demonstrated in the art. By measuring Ang II levels and expression of AGT, renin, and ACE, activation of the circulating and local cardiac RAS is determined

4) Analysis of the established markers of cardiac remodeling, such as ANP, β-MHC, and TGF-β; and newly identified genes that contribute to diabetic cardiomyopathy, such as sarcoendoplasmic reticulum Ca²⁺ATPase (SERCA2), p90 ribosomal S6 kinase (p90RSK), and prorenin converting enzyme (PRECE)

ANP, β-MHC, SERCA2, p90RSK, and PRECE. ANP and β-MHC are established markers of cardiac remodeling. Decreased expression of SERCA2 has been shown to cause alteration in Ca²⁺ homeostasis responsible for diastolic dysfunction in diabetes. More recently, p90RSK and PRECE have been strongly implicated in diabetic cardiac dysfunction.

5) Analysis of pro-inflammatory cardiac cytokines (TNF-α, IL6, and IL1-β): One of the abnormalities associated with diabetic cardiomyopathy is cardiac inflammation, which is accompanied by elevated levels of cytokines, such as TNF-α, IL6, and IL1-β. Normalization of the cytokine levels by test agents can indicate improvement in cardiac function.

6) Measurement of tissue and cellular concentration of the test agents in the heart: Since aliskiren can produce cardioprotective effects by inhibiting local generation of Ang II in the heart, it is desirable to determine the cardiac concentration of aliskiren in animals treated with this agent.

Several parameters have been included in this study, which are modulated by Ang II. Though some of the above parameters are evident early in diabetes (1 wk), longer studies (4 and 8 wk) would likely differentiate the effects of different RAS blockers more convincingly.

It has surprisingly been found that renin inhibitors may be used for the treatment of diabetic cardiomyopathy.

Specifically, the present inventors have found that renin inhibitors, such as aliskiren, either alone or in combination, can have a beneficial effect in the treatment of diabetic cardiomyopathy.

iAng II has been shown to produce multiple biological actions, including cardiac hypertrophy (Kumar R at al in Trends Endocrinol Metab 18:208-214, 2007; Baker K M at al in Regul Pept 120:5-13, 2004). It has been found, by the use of a STZ-induced diabetes in mice (representative of type-1 diabetes), that there is activation of the intracellular renin-angiotensin system (RAS) in diabetic rat hearts. The present invention shows that hyperglycemia activates the cardiac intracellular renin-angiotensin system in vivo and demonstrates that iAng II (intracellular Ang II) participates in the development of cardiovascular pathological conditions associated with diabetes. Significantly, it has been found that blockade of the RAS by a renin inhibitor, in diabetic rats, provides greater protection from cardiac fibrosis and oxidative stress, compared to inhibition with an AT₁ antagonist or an ACE inhibitor. For example, iAng II synthesis is blocked by aliskiren but, not benazepril and, diabetes-induced cardiac fibrosis is partially inhibited by candesartan and benazepril, whereas aliskiren produces complete inhibition. Renin inhibitors provide thus a significant cardiovascular benefit in diabetic conditions.

High glucose is the major stimulus for activation of the intracellular RAS. Both type 1 and type 2 diabetes are accompanied by hyperglycemia. Thus, the observations regarding the intracellular RAS in type 1 diabetes can apply to type 2 diabetes.

This assumption is supported by the results of in vitro studies that have demonstrated activation of the intracellular RAS in neonatal rat cardiac myocytes by high glucose even in the presence of insulin. Furthermore, histological analysis of cardiac tissue from type 2 diabetes patients has shown enhanced intracellular staining for Ang II (Frustaci A at al in Circ Res 87:1123-1132, 2000).

The pharmaceutical activities as effected by administration of a renin inhibitor or by administration of a combination of therapeutic agents used according to the present invention may be demonstrated, e.g., by using corresponding pharmacological models well-known in the pertinent art. A person skilled in the art is fully enabled to select a relevant test model to prove the hereinbefore and hereinafter indicated therapeutic indications and beneficial effects.

A combination according to the present invention comprising a renin inhibitor, or a pharmaceutically acceptable salt thereof, can be administered by various routes of administration. Each agent can be tested over a wide-range of dosages to determine the optimal drug level for each therapeutic agent in the specific combination to elicit the maximal response. For these studies, it is preferred to use treatment groups consisting of at least 6 animals per group. Each study is best performed in a way wherein the effects of the combination treatment group are determined at the same time as the individual components are evaluated.

The usefulness of a renin inhibitor, either alone or in combination with a further active ingredient as described herein, for the treatment of diabetic cardiomyopathy may be demonstrated experimentally, for example, by carrying out a study with C57/BL6 mice and by measuring the parameters hereinafter.

### Methods

### Abbreviations

RAS=renin-angiotensin system
AGT=angiotensinogen
Ang II=angiotensin
iAng II=intracellular Ang II
ARB=angiotensin receptor blocker
WT=wild type
IVSTd=end-diastolic interventricular septum
IVSTs=end-systolic interventricular septum
PWTd=posterior wall thickness diastolic
PWTs=posterior wall thickness systolic
AWTd=anterior wall thickness
LVDd=left ventricular internal diameters diastolic
LVDs=left ventricular internal diameters systolic
FS=fractional shortening
RWT=relative wall thickness
LV=left ventricle
LVM=left ventricular mass
dP/dt=first derivative of left ventricular (LV) pressure over time
PBS=phosphate buffered saline
DNA=deoxyribonucleic acid
DHE=dihydroethidium
ROS=reactive oxygen species

### ELISA=Enzyme-Linked ImmunoSorbent Assay

RT-PCR=reverse transcriptase-polymerase chain reaction
AGT=angiotensinogen
ACE=angiotensin converting enzyme
TGF-β=transforming growth factor-β
ANP=atrial natriuretic peptide
β-MHC=β-myosin heavy chain
SERCA2=sarco-endoplasmic reticulum Ca2+-ATPase
p90RSK=p90 ribosomal S6 kinase
PRECE=prorenin converting enzyme
GAPDH=Glyceraldehyde 3-phosphate dehydrogenase
RNase=ribonuclease
RNA=ribonucleic acid
cDNA=complimentary DNA
TNF-α=Tumor necrosis factor-α
IL6=interleukin 6
IL1-β32 interleukin 1-β
STZ=streptozotocin
s.c.: subcutaneous
b.i.d. (administration): bis in a day
t.i.d. (administration): tris in a day
wk: week
HbA1c=hemoglobin A1c
HF=heart failure

The following study procedures are used:

### Method 1

### Type 1 Diabetes:

Eight groups of eight wk old male mice C57/BL6 are utilized: control, diabetic, and control and diabetic mice treated with an angiotensin II receptor antagonist, for example, candesartan, with an ACEI, for example benazepril, and with a renin inhibitor, for example aliskiren. Each group includes 15 animals. Diabetes is induced by multiple intraperitoneal injections of low dose (for example 50 mg/kg/day for 5 days) streptozotocin (Sigma) in 10 mM citrate buffer. Animals in control groups receive buffer alone. This protocol produces robust and consistent hyperglycemia in mice. Establishment of diabetes is confirmed by blood glucose measurements of >250 using a glucometer. Blood glucose levels are monitored twice a week to confirm sustained diabetes. A control diabetic group in which glucose levels are normalized by subcutaneous insulin treatment is also included. The angiotensin II receptor antagonist, for example candesartan, the ACEI, for example benazepril, and the renin inhibitor, for example aliskiren is administered by osmotic minipump. The animals are studied for cardiac function, morphology, histology, and gene expression at 1, 4 and 8 weeks of establishment of diabetes. The osmotic minipumps are replaced after 4 weeks in 8 week groups. At these time points, significant effects of diabetes on cardiac systolic and diastolic function, fibrosis, oxidative stress, and gene expression are determined.

### Type 2 Diabetes:

Eight groups of six wk old male mice, diabetic (db/db) and non-diabetic littermate control on a C57BLKS/j background are utilized: control, diabetic, and control and diabetic mice treated with an angiotensin II receptor antagonist, for example, candesartan; with an ACEI, for example benazepril; and with a renin inhibitor, for example aliskiren. Each group includes 15 animals. Diabetes is confirmed by blood glucose measurements of >250, using a glucometer. The angiotensin II receptor antagonist, for example candesartan; the ACEI, for example benazepril; and the renin inhibitor, for example aliskiren are administered by osmotic minipump. The animals are studied for cardiac function, morphology, histology, and gene expression following 1, 4 and 8 weeks of treatment. The osmotic minipumps are replaced after 4 weeks in 8 week groups. At these time points, the effects of treatment on diabetes-induced cardiac systolic and diastolic function, fibrosis, oxidative stress, and gene expression are determined.

### Parameters Studies:

### i) Morphometric and Hemodynamic Analyses:

Systolic blood pressure is determined weekly by tail-cuff plethysmography. Echocardiographic analysis are performed on anesthetized animals (with for example 40-50 mg/kg ketamine and for example 5 mg/kg xylazine intraperitoneally) using an Agilent 5500 Sono S echocardiograph equipped with a 12 MHz transducer. After obtaining two-dimensional short-axis images of the left ventricle at the level of the papillary muscle. M-mode freeze frames are obtained. End-diastolic and end-systolic interventricular septum (IVSTd, IVSTs), posterior wall thickness (PWTd, PWTs), anterior wall thickness (AWTd) and left ventricular internal diameters (LVDd, LVDs) are measured using a computer analysis system. Percent fractional shortening (% FS), relative wall thickness (RWT), and left ventricular mass (LVM) are calculated using standard formulas in the art. Echocardiographic analyses are performed the day of the first streptozotocin injection and weekly thereafter. A group of 6 mice are anesthetized at 1, 4, and 8 wk after diabetes induction, with 40-50 mg/kg of for example ketamine and 5 mg/kg of for example xylazine, intraperitoneally. Inotropic and lusitropic function are evaluated by measuring the maximum rate of left ventricular pressure developed (dP/dt max) and left ventricular pressure decay (dP/dt min) with a micromanometer catheter (Millar 1.4 F. SPR 671, Millar Instruments, Texas), positioned in the left ventricle via right common carotid artery cannulation. Mice are killed, and hearts excised, weighed, and processed for histological analyses.

### (ii) Histological Analysis for Fibrosis:

Histological analysis are performed using the hearts obtained in the above study (i) at 1, 4, and 8 wk after diabetes induction. Excised hearts are rinsed in PBS, followed by incubation in Krebs-Hanseleit solution, lacking Ca2+, to relax the cardiac muscle before fixation in 10% formalin. After dehydration in ethanol, and mounting in paraffin, 5 μm thick sections are cut. Sections are stained with hematoxylin and eosin for morphological analysis, and with picrosirius red (Fluka) for detection of fibrosis. To measure the myocyte area, cross-sections with nearly circular capillary profile and nuclei are used, from 10 separate sections.

iii) Cardiac Myocyte Apoptosis:

To determine if there is an increase in the number of TUNEL-positive cardiac myocytes in these hearts, 5 μm thick paraffin sections are deparaffinized by immersing in xylene, rehydrated, and incubated with proteinase K (20 μg/ml). After inactivation of endogenous peroxidases with 3% H₂O₂ in methanol, sections are incubated with terminal deoxynucleotidyl transferase and biotinylated deoxyuridine 5-triphosphate, by using the In Situ Cell Death Detection Kit (Roche). The labeling is detected by streptavidin-HRP and diaminobenzine and observed microscopically. As a positive control, sections are pretreated with micrococcal Dnase I (1 mg/ml) to induce DNA strand breaks. Sections of the heart are co-stained for cardiac myocyte-specific sarcomeric α-actinin with the monoclonal antibody EA-53 (Sigma), to distinguish cardiac myocytes from fibroblasts. An average of 1000 EA-53 positive cells, from random fields, are analyzed.

iv) ROS measurement are performed in frozen heart sections by staining with dihydroethidium (DHE), a cell-permeable fluorescent dye that is oxidized by superoxide to ethidium bromide, which is trapped intracellularly by intercalation into the DNA. Frozen heart sections (20 μM) are incubated with 10 μM DHE at 37° C. for 45 min in a humidified chamber protected from light. Fluorescent images of intercalated dye are obtained using a fluorescent microscope.
v) iAng II Measurement:

Mice are anesthetized and treated with heparin (5000 units/Kg body weight, IP), 10 min prior to harvesting hearts. The chest are opened at the sternum and the heart cannulated with a 20 G phalanged stainless steel cannula into the ascending aorta, and quickly removed. The heart are perfused retrograde through the aorta, using in a Krebs-Henseleit buffer at a constant pressure of 80 mm Hg. Collagenase solution (0.1% w/v) are added to the perfusion buffer and hearts perfused for 45 min. After perfusion, ventricles are out into small pieces and transferred to a spinner flask containing collagenase solution. Dispersed cells are harvested by decantation, after each 5 min of incubation. Myocytes are separated from non-myocytes on a discontinuous percoll gradient. Ang II is extracted from the purified cells and concentration determined by ELISA.

### vi) Gene Expression:

Real time RT-PCR are used to measure expression of AGT, renin, ACE, TGF-β, ANP, β-MHC, SERCA2, p90RSK, and PRECE. GAPDH are measured as a housekeeping gene for relative quantification. Primers and probes are synthesized as described in the literature, for example as described in Naito et al, Hypertension, 2002, 40, 827; Itoh et al Circulation, 2006, 113, 1787 and in Hu et al, Circulation Research, 2005, 96, 1006. Briefly, hearts are washed in PBS and quickly transferred to RNase Later (Ambion) solution for storage at −80° C. RNA isolation (ToTally RNA kit from Ambion) and cDNA synthesis (H)gh capacity cDNA reverse transcription kit from Applied Biosystems) are performed using commercially available kits.

vii) Protein Expression:

Expression of the above genes are confirmed at protein level by Western analysis. Proteins are separated by polyacrylamide gel electrophoresis and transferred to a nitrocellulose membrane. The membrane is probed with a primary antibody specific to the target protein. A secondary antibody, labeled for detection with either chemiluminescence or fluorescence, is used to quantitate the primary antibody bound to the membrane, which corresponds to the amount of the target protein.

vii) Cytokines:

Pro-inflammatory cardiac cytokines TNF-α, IL6, and IL1-β are measured by commercially available ELISA kits (Pharmingen/BD Biosciences), according to the manufacturer's instructions (Westermann et al Diabetologia 2006, 49, 2507).

The experimental study can be summarized as follows:

| | | | Duration of | |
|---|---|---|---|---|
| Treatment | | N/group | Treatment (weeks) | Relevant Information |
| No | Control | 15 | 1, 4, and 8 | Morphometric, |
| treatment | Diabetic | 15 | | hemodynamic parameters |
| Candesartan | Control | 15 | 1, 4, and 8 | are measured in all animals. |
| | Diabetic | 15 | | 6 animals from each group |
| Benazepril | Control | 15 | 1, 4, and 8 | are used for histology-based |
| | Diabetic | 15 | | parameters. |
| Aliskiren | Control | 15 | 1, 4, and 8 | 6 animals are used for gene |
| | Diabetic | 15 | | and protein expression |
| | | | | studies and Ang II |
| | | | | measurement. |
| | | | | 3 extra animals are included |
| | | | | to account for mortality due |
| | | | | to diabetes or handling. |
| Insulin | Diabetic | 15 | 1, 4, and 8 | Control to exclude toxic |
| | | | | effects of STZ |

### Method 2

### Induction of Diabetes and Treatment of Animals

Diabetes is induced by a single injection of streptozotocin (STZ, 65 mg/kg body weight, IP) dissolved in 0.1 M sodium citrate buffered saline (pH 4.5), in adult male Sprague Dawley rats (250-300 g). Control animals receive buffered saline alone. Diabetes is confirmed by sustained blood glucose levels >15 mmol/L, as determined 48 h after STZ injection and on alternate days thereafter. Diabetic rats, in groups of nine animals, are treated with either insulin (2-5 U, BID, SC), treated with an angiotensin II receptor antagonist, for example, candesartan (1 mg/kg, IP), with a renin inhibitor, for example aliskiren (30 mg/kg, oral), or with an ACEI, for example benazepril (10 mg/kg, oral), daily for 7 days, beginning 48 h after STZ injection. After 7 days, animals are weighed and anesthetized using ketamine/xylazine (50/5 mg/kg). Hearts are isolated and weighed before perfusion, the latter using the Langendorff methodology.

Isolation of Cardiac Myocytes and Measurement of iAng II

Hearts are isolated and perfused with Krebs-Henseleit bicarbonate buffer, followed by digestion with collagenase II (0.1% w/v). Myocytes are separated from non-myocytes by differential centrifugation at 25×g. The purity of the myocyte preparations using this procedure is >90%, as analyzed by FACS, using anti-sarcomeric myosin (MF-20) and anti-sarcomeric actin antibody. The pellet containing myocytes is processed for Ang II extraction, as described by Singh et al in Am J Physiol Heart Circ Physiol 293:H939-H948, 2007. Cells were lysed in ice-cold 1 M acetic acid, containing a protease inhibitor cocktail (Sigma), by sonication (2 pulses of 5 sec each). The lysate is sedimented at 20,000×g for 10 min and the supernatant is dried in a vacufuge, followed by reconstitution in 1% acetic acid. The samples are applied to a conditioned DSC-18 column (Supelco), washed, and eluted with methanol. The eluted samples are dried and reconstituted in PBS for ELISA. For isolation of Ang II from plasma, an equal volume of 2% acetic acid is added to plasma, followed by filtration through Amicon Ultra-15 filters. The filtrate is applied to DSC-18 columns and Ang II is eluted, as described for the cell lysates. Using the above procedure, >90% recovery of exogenously added Ang II can be recovered. Ang II is measured by quantitative, competitive ELISA, using a specific anti-Ang II antibody (Peninsula Labs), as previously described in Am J Physiol Heart Circ Physiol 293:H939-H948, 2007. ELISA is performed on protein-A and anti-Ang II antibody-coated 96-well dishes. Competitive binding of synthetic biotinylated Ang II, in the presence of the extracted peptide, is detected with streptavidin-horseradish peroxidase conjugate. A standard curve, generated from binding of a constant amount of biotinylated Ang II with increasing concentrations of non-biotinylated synthetic Ang II, is used to calculate the concentration of the peptide in the sample. The concentration of Ang II in the cell lysates is expressed as fmoles per milligram of heart wt and in plasma as fmoles per milligram of plasma proteins.

Ang II levels in cardiac myocytes, which are isolated after perfusion of the hearts and enzymatic dispersion, correspond to Ang II present intracellularly. To determine the source of iAng II, i.e., intracellular synthesis or AT₁-mediated internalization, one group of diabetic animals is treated with the AT₁ antagonist candesartan to prevent receptor-mediated uptake. Cardiac myocytes from diabetic rat hearts show a 9.9-fold elevation in the levels of iAng II (0.59±0.01 fmole/mg heart wt), compared to cells from control animals (0.06±0.01 fmole/mg heart wt). Normalization of blood glucose levels by insulin, in STZ-treated rats, completely blocks the rise in iAng II levels (0.16±0.02 fmole/mg heart wt), indicating that the latter is a specific effect of hyperglycemia. Treatment of diabetic rats with candesartan, reduces iAng II levels to 0.43±0.05 fmole/mg heart wt, which indicates that the major source of iAng II is intracellular synthesis. Treatment of diabetic rats with aliskiren normalizes iAng II levels (0.12±0.02 fmole/mg heart wt), while benazepril does not have any effect (0.55±0.02 fmole/mg heart wt.

### Cardiac Myocyte Apoptosis

Apoptotic cardiac myocytes are detected in paraffin-embedded heart sections using the terminal deoxynucleotide transferase-mediated dUTP nick end labeling (TUNEL) assay and cleaved caspase-3 staining. TUNEL assay is performed using an assay kit (Millipore Corporation, Temecula, Calif.), as per the manufacturer's instructions. Cytoplasm and nuclei from the myocytes are counter-stained using α-sarcomeric actin antibody and DAPI, respectively. For cleaved caspase-3 staining, deparaffinized sections are subjected to antigen retrieval in 0.01 M citrate buffer (pH 6.0), by microwaving. After blocking with 5% BSA, the sections are incubated with rabbit monoclonal anti-cleaved caspase-3 antibody (1:200; Cell Signaling Technology, Danvers, Mass.) overnight at 4° C., followed by fluorescein isothiocyanate-conjugated goat anti-rabbit IgG (1:200; Molecular Probes). The number of positively stained nuclei is counted from twenty fields/heart and three hearts/treatment group.

Quantification of apoptotic cells shows a 3-fold increase in diabetic hearts, compared to control, by both TUNEL assay and caspase-3 staining. Normalization of blood glucose by insulin or blockade of the RAS with the three different inhibitors, reduces the number of apoptotic cells, but does not prevent apoptosis completely.

| | |
|---|---|
| Caspase-3 | TUNEL |
| staining | assay |

(positive cells/mm²)

| | Mean | SE | Mean | SE |
|---|---|---|---|---|
| Cont | 7 | 0.16 | 8.5 | 0.34 |
| Diabetic | | | | |
| No treatment | 28 | 0.35 | 33 | 0.7 |
| Ins | 14 | 0.18 | 20 | 0.21 |
| Alsk | 18 | 0.48 | 21.3 | 0.83 |
| Cand | 18.5 | 0.42 | 24 | 0.34 |
| Bnz | 21 | 0.28 | 27 | 0.6 |

### Reactive Oxygen Species (ROS) Detection in the Heart

Superoxide production in the hearts is detected by dihydroethidium staining (DHE, Sigma-Aldrich). Frozen heart sections (20 μm thick) are incubated with 10 μM DHE, at 37° C. for 45 min, in a humidified chamber protected from light. Fluorescent images, obtained with an Olympus FV300 confocal microscope, are analyzed with Slide Book 4.2. The mean DHE fluorescence intensity of myocyte nuclei is calculated by dividing the combined fluorescence value of the pixels by the total number of pixels, in fifteen randomly selected fields observed with identical laser and photomultiplier settings. Diabetic hearts show enhanced superoxide production, compared to control animals, which is prevented in insulin treated animals. Treatment of diabetic rats with candesartan or benazepril, reduces oxidative stress, while aliskiren blocks it completely.

DHE fluorescence
(Arbitrary units)

| | Mean | SE |
|---|---|---|
| Cont | 17.65 | 0.5 |
| Diabetics | | |
| No treatment | 68.6 | 1.9 |
| Ins | 26.7 | 2 |
| Alsk | 22.7 | 0.35 |
| Cand | 37.7 | 1.3 |
| Bnz | 51.4 | 1.9 |

### Cardiac Fibrosis

Cardiac interstitial fibrosis is determined by Masson's trichrome staining on 5 μm paraffin-embedded sections. The extent and degree of fibrosis is graded on a scale of 0-4. Grade 0 signifies no apparent collagen fiber proliferation except for small islets of fibrous tissue around the capillaries, as well as an intercellular single layer of collagenous tissue, as in normal myocardium. Focal and minimal fibrosis is graded as 1, mild patchy fibrosis as grade 2, moderate diffuse fibrosis as grade 3 and the most prominent fibrosis, covering major area of the specimen, is classified as 4. A minimum of three sections per heart, with five fields per section, and three animals per experimental group, are analyzed and results presented as an average grade.

After one week of diabetes, the overall staining for fibrosis is enhanced in hearts from diabetic rats (grade 1.5), compared to control animals (grade 0). Insulin treatment completely prevents the increase in fibrosis (grade 0.04). Candesartan and benazepril reduces the degree of fibrosis (grade 0.43 and 0.88, respectively), whereas aliskiren has a more pronounced reduction of fibrosis (grade 0.25) in diabetic rat hearts.

### Statistical Analysis

Values are expressed as the means±SE. ANOVA with Tukey's post hoc test was used for statistical analysis. P<0.05 is considered statistically significant.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent.

The above results demonstrate that renin inhibitors, such as aliskiren, either alone or in combination with other active agents can be useful in the treatment of diabetic cardiomyopathy.

1. A method of using a renin inhibitor or a pharmaceutically acceptable salt thereof to treat diabetic cardiomyopathy comprising administering a therapeutically effective amount of the renin inhibitor or pharmaceutically acceptable salt thereof to a patient in need of such treatment.

2. The method according to claim 1 for the treatment of type 2 or type 1 diabetic patients.

3. The method according to claim 1 wherein the renin inhibitor is aliskiren or a salt thereof.

4. A pharmaceutical composition for the treatment of diabetic cardiomyopathy comprising a renin inhibitor or a pharmaceutically acceptable salt thereof.

5. The pharmaceutical composition according to claim 4 for the treatment of type 2 or type 1 diabetic patients.

6. The pharmaceutical composition according to claim 4 wherein the renin inhibitor is aliskiren or a salt thereof.

7. The method according to claim 1 wherein the renin inhibitor or a pharmaceutically acceptable salt thereof is used in combination with one or more additional active ingredient.

8. The method according to claim 7 wherein the additional active ingredient is selected from the group consisting of ACEIs, beta blockers, angiotensin II receptor antagonists, type 2 diabetes therapeutic agents, and type 1 diabetes therapeutic agents, or in each case independently a salt thereof.

9. The method according to claim 7 wherein the other active ingredient is
an ACEI selected from the group consisting of alacepril, benazepril, benazeprilat, captopril, ceronapril, delapril, enalapril, enaprilat, fosinopril, imidapril, lisinopril, moveltopril, perindopril, quinapril, ramipril, spirapril, temocapril and trandolapril, or in each case independently a salt thereof;
an angiotensin II receptor antagonist selected from the group consisting of valsartan, losartan, eprosartan, irbesartan, telmisartan, candesartan and saprisartan, or in each case independently a salt thereof;
a type 2 diabetic therapeutic agent selected from the group consisting of troglitazone, rosiglitazone, ciglitazone; darglitazone; englitazone; isaglitazone and pioglitazone, or in each case independently a salt thereof; and/or
a type 1 diabetic therapeutic agent, such as insulin, or a salt thereof.

10. The method according to claim 7 for simultaneous, separate or sequential use.

11. A pharmaceutical composition according to claim 4 comprising of an additional active ingredient.

12. A pharmaceutical composition according to claim 11 wherein the additional active ingredient is selected from the group consisting of ACE inhibitors, angiotensin II receptor antagonists, beta-blockers, type 2 diabetes therapeutic agents, and type 1 diabetes therapeutic agents.

13. A pharmaceutical composition according to claim 11 wherein the further active ingredient is
an ACEI selected from the group consisting of alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril and trandolapril, or in each case independently a salt thereof;
an angiotensin II receptor antagonist selected from the group consisting of valsartan, losartan, eprosartan, irbesartan, telmisartan, candesartan and saprisartan, or in each case independently a salt thereof;
a type 2 diabetic therapeutic agent selected from the group consisting of troglitazone, rosiglitazone, ciglitazone; darglitazone; englitazone; isaglitazone and pioglitazone, or in each case independently a salt thereof; and/or
a type 1 diabetic therapeutic agent, such as insulin, or a salt thereof.

14. A commercial package comprising a pharmaceutical composition according to claims 4, together with instructions for simultaneous, separate or sequential use thereof in the treatment of diabetic cardiomyopathy.

15. A kit for the treatment of diabetic cardiomyopathy, which comprises:
a) a renin inhibitor or a pharmaceutically acceptable salt thereof in a first unit dosage form;
b) at least one therapeutic agent selected from the group consisting of an ACE inhibitor, an angiotensin II receptor antagonist (e.g., valsartan), a beta blocker, a type 2 diabetes therapeutic agent, a type 1 diabetes therapeutic agent, or in each case independently a pharmaceutically acceptable salt thereof, in a second etc. unit dosage form;
c) a container for containing said first, second etc. unit forms.

16. A method for the treatment of diabetic cardiomyopathy, which comprises administering to a warm-blooded animal, a therapeutically effective amount of a renin inhibitor, either alone or in combination with one or more active ingredient, selected from the group consisting of ACEIs, beta blockers, angiotensin II receptor antagonists, type 2 diabetes therapeutic agents, type 1 diabetes therapeutic agents, or in each case independently a salt thereof.

17. The method of claim 8 wherein the type 1 diabetes therapeutic agent is insulin.

18. The method of claim 8 wherein the type 2 diabetes therapeutic agent is a TZD.

19. A commercial package comprising a pharmaceutical composition according to claim 11, together with instructions for simultaneous, separate or sequential use thereof in the treatment of diabetic cardiomyopathy.

20. The method of claim 12 wherein the type 1 diabetes therapeutic agent is insulin.

21. The method of claim 12 wherein the type 2 diabetes therapeutic agent is a TZD.
